(19) United States
(12) Patent Application Publication
(10) Pub. No.: US 2011/0177171 A1
(43) Pub. Date: Jul. 21, 2011

(54) Oxygenated Ophthalmic Compositions and Methods of Use

(76) Inventor: Emil W. Chynn, New York, NY (US)

(21) Appl. No.: 13/073,995

(22) Filed: Mar. 28, 2011

Related U.S. Application Data
(62) Division of application No. 11/844,930, filed on Aug. 24, 2007, now abandoned.
(60) Provisional application No. 60/823,809, filed on Aug. 29, 2006.

(51) Int. Cl. A61K 33/00; A61P 27/02; A61P 31/00; A61P 29/00

(52) U.S. Cl. 424/600

(57) ABSTRACT

The present invention relates to oxygenated ophthalmic topical compositions and methods of administering oxygenated ophthalmic topical compositions. The present invention also encompasses methods for making and administering the oxygenated ophthalmic topical compositions for use in treating or ameliorating pathologic conditions, complications, diseases and symptoms related to hypoxia of the anterior eye structure, including corneal neovascularization (CNV), edema, abrasions, infections, infiltrates, dry eye syndrome, contact lens intolerance; and redness, blurred vision, discomfort, foreign body sensation, photophobia, and irritation.

### CROSS-REFERENCE TO RELATED APPLICATIONS

This application is a divisional of and claims priority to co-pending U.S. Application No. 111844,930, filed Aug. 24, 2007, which is the non-provisional of and claims priority to U.S. Provisional Application No. 60/823,809, filed on Aug. 29, 2006, both of which are incorporated by reference in its entirety.

### FIELD OF THE INVENTION

The present invention relates to oxygenated ophthalmic compositions and methods of administering oxygenated ophthalmic compositions. The present invention also encompasses methods for making and administering the oxygenated ophthalmic compositions for use in treating various ocular complications, conditions, and diseases related to ocular hypoxia.

### BACKGROUND OF THE INVENTION

Tear substitutes, more commonly referred to as artificial tears, are used in the treatment of diseases or conditions of the eye, such as Dry Eye Syndrome (DES) or keratoconjunctivitis sicca (KC), which can be due to many different causes. Some causes of these diseases or conditions include reduction in tear production in the elderly, rheumatic disease (such as polyarthritis nodosa), diabetes and thyroid disease, autoimmune disease (Sjogren's disease, Lupus, scleroderma), skin diseases, hormonal changes, neuroparalysis (such as after a stroke, defective position of the eyelid, decortication of tear glands), ingestion of certain drugs (such as beta blockers, birth control pill, soporifics, antihistamines, and tranquilizers), nutritional deficiency (such as Vitamin A), climatic influences (eat, dry environmental air, air conditioners), environmental pollution (ozone, dust, solvent vapors, etc.), and poor workplace habits (staring at a computer monitor for too long without relief). Dry eyes may also be caused by diseases of the hematopoietic system, local eye diseases such as inflammation, and trauma of the tear glands or hereditary diseases. The resulting dysfunctions impair or prevent the formation of a normal tear film.

There are essentially three components of the tear film: an internal mucin layer which covers the epithelial surface, a middle aqueous layer, and a thin, external lipid layer. The mucin layer functions as a corneal wetting agent. The aqueous component has lubricant, cleaning, and protective functions. The lipid component prevents evaporation of the aqueous component and prevents runoff of the tear film.

An intact tear film is necessary to maintain good ocular health. Dry eye syndrome is caused by either deficient tear production or excessive tear evaporation or some combination of the two. DES can lead to significant symptoms (e.g., redness, irritation, foreign body sensation, etc.) in the short-term and pathology (superficial punctuate keratopathy, epithelial erosions, sub-epithelial scarring, etc.) in the long-term, and thus requires treatment, the most common of which is the instillation of artificial tears.

Conventional artificial tears that are used to treat dry eyes typically contain one or more of the following lubricating agents: Hydroxypropyl Methylcellulose (HPMC), Carboxyl Methylcellulose (CMC), Polyvinyl Alcohol (PVA), Glycerin, hyaluronidase, or other similar agents. More recent artificial tear compositions attempt to more closely replicate the composition of natural human tears, by containing, in addition to water, electrolytes, small molecules such as carbohydrates and lipids, and proteins. Many artificial tears also include hydrogels to enhance viscosity to improve retention time on the corneal surface. In addition, many artificial tears contain preservatives to increase longevity and shelf-life, although preservatives are avoided in certain “preservative free” artificial tears that come in individual vials.

Artificial tears are often hypotonic with relation to natural tears because in a dry eye, the water portion of the tear is often partially evaporated, leaving behind a hypertonic mixture of salts and electrolytes. Thus a solution that is hypotonic with relation to natural tears (i.e., a solution with more solvent (water) than solute (electrolytes, salts, etc)) is necessary to reestablish isotonicity.

Although many kinds of artificial tears are relatively effective in treating dryness as well as ocular hypertonicity, a major co-morbid factor that is often present in DES—namely, ocular hypoxia—remains untreated by tear formulations currently on the market.

Irrigating solutions are those used during intraocular procedures such as cataract and retinal surgery. One investigator has examined whether oxygenating irrigating solutions during intraocular surgery can potentially enhance postoperative recovery (U.S. Pat. No. 6,585,679). However, such irrigating solutions are only used intraocularly during surgery, not topically, and thus only improve oxygenation to the posterior eye structures, not the anterior eye structures, and are not useful outside of a surgical setting.

Presently there are no oxygenated ophthalmic compositions for use to increase the oxygen concentration of the tear film, and no oxygenated compositions for treating or preventing hypoxia-induced damage to the anterior structures of the eye. Thus, there is a need for compositions and methods to increase the oxygen concentration of the tear film, thus treating or preventing damage to the superficial anterior structures of the eye.

### SUMMARY OF THE INVENTION

The present invention provides oxygenated ophthalmic compositions and methods for treating various ocular complications, conditions, and diseases related to ocular hypoxia. Additionally, the invention provides methods for making oxygenated ophthalmic compositions. Embodiments of the present invention also relate to kits containing oxygenated ophthalmic compositions.

In certain embodiments, the invention relates to an ophthalmic composition comprising a solution of an aqueous ophthalmic agent and oxygen. In certain embodiments the oxygen is in gaseous form. In additional embodiments, the solution is substantially oxygenated.

In certain embodiments, the aqueous ophthalmic agent is artificial tears or an aqueous solution of an ophthalmic agent selected from the group consisting of an antibacterial agent, an antiviral agent, a corticosteroid, a dilating agent, a glaucoma agent, a mydriatic, a cycloplegic, a non-steroidal anti-inflammatory drug (NSAID), ocular antihistamine, ocular decongestant, ocular MAST cell stabilizer, allergy agent, other medicated eye drop, and combinations thereof.

In certain embodiments, the artificial tears further comprise a diluting agent and a wetting agent. In certain embodiments, the diluting agent is water, distilled water, or sterile water.

In certain embodiments, the wetting agent is selected from the group consisting of carboxymethylcellulose (CMC), hydroxypropyl methylcellulose (HPMC), glycerin, mannitol, polyvinyl alcohol (PVA), hydroxyethylcellulose, Carbopol, polyvinyl pyrrolidone, polyethylene glycol, dextran, hyaluronic acid, carbomer 940 (polyacrylic acid), and combinations thereof and wherein the concentration of the wetting agent is between about 0.001 and about 10 volume percent based on the total volume of the artificial tears.

In certain embodiments, the ophthalmic composition further comprises a diagnostically acceptable amount of an excipient. In additional embodiments, the excipient concentration is between about 0.01 and about 0.03 volume percent based on the total volume of the aqueous solution. In yet additional embodiments, the excipient is selected from the group consisting of an antioxidant, sodium bisulfate, metabisulfite, hydrochloric acid, and sodium hydroxide.

In additional embodiments, the ophthalmic composition farther comprises a diagnostically acceptable amount of an additive. In certain embodiments, the additive is a buffer. In certain embodiments, the buffer is sodium bicarbonate or sodium lactate. In certain embodiments, the pH of the composition is between about 4.0 and about 7.5.

In certain embodiments, the ophthalmic composition farther comprises a diagnostically acceptable amount of a salt to match the ocular electrolytic balance. In certain embodiments, the salt is selected from the group consisting of calcium chloride, magnesium chloride, potassium chloride, sodium chloride, sodium phosphate, and combinations thereof. In additional embodiments, the salt is present in a concentration between about 0.01 and about 4.0 volume percent based on the total volume of the aqueous ophthalmic agent.

In certain embodiments, the ophthalmic composition farther comprises a preservative. In additional embodiments, the preservative is selected from the group consisting of sodium perborate, benzalkonium chloride, purite, polyquaternium-1, sodium silver chloride complex, polyhexamethylene biguanide, sorbic acid, chlorobutanol, a stabilized oxyborate complex, and combinations thereof. In additional embodiments, the preservative is present in a concentration between about 0.001 and about 1.0 volume percent based on the total volume of the solution.

In certain embodiments, the invention relates to a method of treating an ocular condition comprising administering an ophthalmic composition comprising a solution of an aqueous ophthalmic agent containing oxygen via topical ocular administration to a patient in need thereof.

In certain embodiments, the aqueous ophthalmic agent is artificial tears or an aqueous solution of an ophthalmic agent selected from the group consisting of an antibacterial agent, an antiviral agent, a corticosteroid, a dilating agent, a glaucoma agent, a mydriatic, a Cycloplegic, a non-steroidal anti-inflammatory drug (NSAID), ocular antihistamine, ocular decongestant, ocular MAST cell stabilizer, allergy drop, other medicated drop, and combinations thereof.

In certain embodiments, the ocular condition comprises one or more of the conditions selected from the group consisting of dry eye, corneal redness, itching, edema, abrasions, and infiltrates, ocular swelling redness, and irritation, blurred vision, discomfort, night halos, contact lens intolerance, corneal neovascularization, hypoxia in and around the eye, and combinations thereof.

In certain embodiments, the invention relates to a method of oxygenating aqueous ophthalmic composition comprising the steps of: contacting an aqueous ophthalmic solution with oxygen gas; filling a single-dose vial with the oxygenated aqueous ophthalmic composition; and sealing the single-dose vial while maintaining an oxygen overpressure.

In certain embodiments, the oxygen is contacted with the aqueous ophthalmic solution by one or more of the oxygenating techniques selected from the group consisting of bubbling, spraying, violent shaking, aerating, maintaining an oxygen overpressure, electrolysis, cold oxygen plasma exposure, liquid oxygen, diffusion, and combinations thereof.

In certain embodiments, the invention relates to a method of oxygenating an aqueous ophthalmic solution comprising the steps of: partially filling a single-dose vial with an aqueous ophthalmic solution; filling the remainder of the single-dose vial with substantially pure oxygen gas; and sealing the single-dose vial to encapsulate the aqueous ophthalmic solution and oxygen gas.

In certain embodiments, the invention relates to a method of maintaining oxygen saturation in an aqueous ophthalmic solution comprising: filling a single-dose vial with an oxygenated aqueous ophthalmic solution; filling the headspace of the single-dose vial with substantially pure oxygen gas; and sealing the single-dose vial.

In certain embodiments, the invention relates to a method of maintaining oxygen saturation in an aqueous ophthalmic solution comprising filling a single-dose vial with an oxygenated aqueous ophthalmic solution in the presence of an oxygen overpressure and sealing the single-dose vial.

In certain embodiments the invention relates to an oxygenated ophthalmic composition obtained by any of the methods of the present invention.

In certain embodiments, the oxygenated ophthalmic composition is contained in a single-dose vial.

In certain embodiments the invention relates to a kit comprising at least one of the ophthalmic compositions of the present invention.

In certain embodiments the kit further comprises patient instructions and optionally eye wipes, or any suitable eye tissue or wiping means. In certain embodiments the eye wipes, tissues or wiping means are wet, and in certain embodiments they are dry. Additionally, the eye wipes, tissues or wiping means may be individually packaged or packaged in multiples.

### DESCRIPTION OF THE INVENTION

The present invention relates to an ophthalmic solution such as an artificial tear with a high oxygen concentration (PO₂) which would preferably be given to patients in single-dose vials and would increase oxygen delivery to the eye's anterior structures, thereby improving relieving the symptoms and pathology associated with hypoxia of the anterior eye structures (e.g., in patients with dry eye syndrome, contact lens intolerance, etc.) The oxygenated ophthalmic solution can also provide effective amelioration and treatment of ocular pathology including corneal neovascularization (CNV), edema, abrasions, infections, and infiltrates; and ameliorate and/or treat associated symptoms including redness, blurred vision, discomfort, dry eyes, foreign body sensation, photophobia, and irritation

In accordance with the present invention there may be employed conventional terms that are understood in the field to encompass the following meanings or definitions.

About or Approximately

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5 fold, and more preferably within 2 fold, of a value. Unless otherwise stated, the term ‘about’ means within an acceptable error range for the particular value.

Carrier

The term “carrier” refers to a diluent, adjuvant, excipient, or vehicle with which the compound is administered. Such pharmaceutical carriers can be hydrogels as well as sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

Dosage

The dosage of the therapeutic formulation will vary widely, depending upon the nature of the disease, the patient's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level.

Treating or Treatment

“Treating” or “treatment” of a state, disorder or condition includes:

(1) preventing or delaying the appearance of clinical or sub-clinical symptoms of the state, disorder or condition developing in a mammal that may be afflicted with or predisposed to the state, disorder or condition but does not yet experience or display clinical or subclinical symptoms of the state, disorder or condition; or

(2) inhibiting the state, disorder or condition, i.e., arresting, reducing or delaying the development of the disease or a relapse thereof (in case of maintenance treatment) or at least one clinical or sub-clinical symptom thereof; or

(3) relieving the disease, i.e., causing regression of the state, disorder or condition or at least one of its clinical or sub-clinical symptoms.

The benefit to a subject to be treated is either statistically significant or at least perceptible to the patient or to the physician.

Therapeutically Effective Amount

A “therapeutically effective amount” means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

Patient or Subject

“Patient” or “subject” refers to mammals and includes human and veterinary subjects.

Pharmaceutically Acceptable

As used herein, the phrase “pharmaceutically acceptable” refers to molecular entities and compositions that are “generally regarded as safe”, e.g., that are physiologically tolerable and do not typically produce an allergic or similar untoward reaction, such as itching or blurred vision, dizziness and the like, when administered to a patient or to any animal in need of treatment. Preferably, as used herein, the term “pharmaceutically acceptable” means approved by a regulatory agency of the Federal or a state government or listed in the U.S. Pharmacopoeia or other generally recognized pharmacopeias for use in animals, and more particularly in humans.

Pharmaceutical Compositions and Administration

While it is possible to use a composition provided by the present invention for therapy as is, it may be preferable to administer it in a pharmaceutical formulation, e.g., in admixture with a suitable pharmaceutical excipient, diluent or carrier selected with regard to the intended route of administration and standard pharmaceutical practice. Accordingly, in one aspect, the present invention provides a pharmaceutical composition or formulation comprising at least one active composition, or a pharmaceutically acceptable derivative thereof, in association with a pharmaceutically acceptable excipient, diluent and/or carrier. The excipient, diluent and/or carrier must be “acceptable” in the sense of being compatible with the other ingredients of the formulation and not deleterious to the recipient thereof.

The compositions of the invention can be formulated for administration in any convenient way for use in human or veterinary medicine. The invention therefore includes within its scope pharmaceutical compositions comprising a product of the present invention that is adapted for use in human or veterinary medicine.

In a preferred embodiment, the pharmaceutical composition is conveniently administered as an ocular ophthalmic formulation.

Such ocular ophthalmic formulations may be presented for use in a conventional manner with the aid of one or more suitable excipients, diluents, and carriers. Pharmaceutically acceptable excipients assist or make possible the formation of a dosage form for a bioactive material and include diluents, binding agents, lubricants, glidants, disintegrants, coloring agents, and other ingredients. Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may be also used. An excipient is pharmaceutically acceptable if, in addition to performing its desired function, it is non-toxic, well tolerated upon ingestion, and does not interfere with absorption of bioactive materials.

Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

Cells on the corneal surface do not derive their oxygen from the blood stream, as the cornea contains few blood vessels in order to maintain transparency of the ocular media. Instead, oxygen is obtained through atmospheric oxygen dissolved in the normal tear film.

Oxygen is necessary for the healthy function of ophthalmic structures and cells, just as in all somatic cells—for respiration, metabolism, and other cellular functions. Ocular hypoxia is thus an important factor in a number of pathologic ophthalmic conditions or disease states, such as corneal neovascularization, corneal edema, corneal abrasions, and recurrent erosion syndrome, which are associated with such symptoms as irritation, redness, contact lens intolerance, foreign body sensation, blurry vision, and photophobia. Corneal hypoxia has also been associated with an increased susceptibility to infections and other pathologic entities, especially when associated with contact lens use (particularly extended wear lenses—which have been found to increase the risk of bacterial keratitis by a factor of ten).

Contact lens intolerance, or the inability to wear contact lenses for an extended period of time, is due only secondarily to dryness, but primarily to corneal hypoxia. Contact lenses block direct contact between the underlying tear film and the atmosphere. Therefore, in the presence of contact lenses, the majority of oxygen delivered to the cornea, even in the newer high-DK (high oxygen transmissibility) lenses is not from diffusion through the lens, but from exchange of (oxygenated) tear film underneath the lens with lens translation during blinking. Thus, every contact lens has the inherent problem of causing some degree of corneal hypoxia in the corneal epithelial, stromal, and endothelial cells.

In contact lens intolerance syndrome, therefore, the wearer feels pain, discomfort, dryness, irritation, foreign body sensation, redness and/or blurry vision—not only because of dryness, but also because of corneal hypoxia. However, all current therapies only treat the dry eye component, not the hypoxic component

Corneal neovascularization (CNV) is the pathologic ingrowth of vessels from the limbal vascular plexus into the cornea in response to corneal hypoxia, which most often is due to excessive contact lens wear. In fact, it is uncommon to see CNV except in contact lens users. The progression of corneal neovascularization can be arrested and, in some cases, even reversed, by discontinuation of contacts lens use. However, for a variety of reasons (recreational and professional requirements, vanity, etc.), many contact lens users—even those with corneal neovascularization—do not wish to stop wearing lenses. One potential treatment in such cases would be to use the composition, most likely in the form of an oxygenated artificial tear, to provide extra oxygen, and thus treat the corneal neovascularization, thus increasing contact lens tolerance, and avoiding the need to discontinue contact lens use.

Corneal hypoxia can also compromise corneal endothelial cell function, resulting in hypoxia-induced corneal edema. Advanced corneal edema can progress to bullous keratopathy, which is a swelling of the cornea causing loss of transparency, poor vision, and eventually severe. pain. Prolonged corneal hypoxia can kill endothelial cells, resulting in permanent loss of transparency and hence poor vision. The present invention can be used to treat corneal hypoxia, keeping corneal endothelial cells alive, and preserving vision.

Corneal hypoxia can also predispose to, or prevent the healing of corneal abrasions, which are caused by ocular trauma due to a foreign body (often a contact lens). Symptoms of corneal abrasions include foreign-body sensation, pain, and photophobia. Corneal epithelial hypoxia impairs attachment of the epithelium to underlying Bowman's membrane, resulting in both non-healing abrasions and recurrent erosions, both of which when chronic can lead to permanent scarring and loss of vision, and can also predispose to vision-threatening infections. Use of the inventive composition will reduce corneal hypoxia and can be used to treat or prevent abrasions and erosions.

Corneal hypoxia has also been shown to significantly increase the risk of bacterial keratitis, by predisposing to micro-abrasions and disrupting immune function. This association has been clearly proven in the users of extended contact lenses, who have a tenfold increased relative risk of vision-threatening infections. The present invention can be used to prevent or treat bacterial keratitis and other infections, particularly in hypoxic contact lens wearers.

Thus, the inventive composition encompasses using oxygenated topical solutions to treat hypoxia of the anterior eye structures, and any pathologic or symptomatic state associated with said hypoxia.

The present inventive composition and method encompasses increasing oxygen saturation in an ophthalmic solution to varying degrees, including: 1) fully saturated with oxygen at normal atmospheric pressure, 2) super-saturation with oxygen at a pressure above normal atmospheric pressure (overpressure), and 3) hyper-oxygenation.

Various methods to achieve each of the above three states of saturation, the composition of the resultant oxygenated solution, and the means of application of the solution are described below in more detail. However, as used herein with respect to the inventive compositions and methods, all three saturation levels are referred collectively as “oxygenated,” which will be understood to be a “catch-all” phrase used to describe all three states/conditions.

In the present invention, ophthalmic compositions are oxygenated and function to provide additional oxygen perfusion to the anterior structures of the eye (e.g., the lids, lashes, lacrimal system, cornea, conjunctiva, sclera, anterior chamber, and iris) rather than the more posterior structures (e.g., lens and retina). The present invention is also directed to an ophthalmic composition produced by a method that yields a unique composition that is oxygenated, and is capable of being used to prevent, palliate or treat the above conditions caused by ocular hypoxia.

The present invention relates to a method to oxygenate ophthalmic solutions to a high PO₂, and to methods for applying or administering oxygenated solutions topically to the surface of the eye, to prevent, relieve, ameliorate, or treat any one or more of the many pathologic conditions associated with hypoxia of the anterior structures of the eye. A preferred inventive solution is an artificial tear solution that is oxygenated, and also contains a lubricant, an excipient, additives and/or buffers, salts to match the ocular electrolytic balance, possibly a preservative, and a diluting agent to increase volume. As used herein, the term solution is used generically to refer to solutions, mixtures, suspensions, gels, ointments, emollients, and combinations thereof.

The present invention of an oxygenated ophthalmic composition may be applied to any prescription or non-prescription medication available in topical, ocular (drop) form. However, a preferred embodiment of the present invention is its use in an artificial tear, as this is the application where it may be most efficacious from a medical point of view, and appealing from a consumer marketing point of view. Therefore, even though a preferred embodiment is described, it is understood that there are many additional embodiments to which the present invention may be equally applied.

Usage and Administration

The topical administration of this inventive composition to the eye will improve comfort and prevent, ameliorate, or treat ocular pathology such as contact lens intolerance, foreign body sensations, and corneal infections, neovascularization, edema, abrasions, infiltrates, ocular swelling, redness, blurred vision, photophobia, and discomfort.

The inventive composition will provide additional benefits for preoperative and postoperative ophthalmic care because the additional oxygen will improve patient comfort and may reduce the incidence of ocular pathologies, including infection, and may speed or otherwise aid postoperative healing of anterior eye structures.

By placing the inventive composition in sealed, single-dose vials, the oxygen concentration will be maintained at high levels during storage while maintaining sterility, thus providing convenient release of the oxygenated product for patient use. The use of an air-tight, multiple-use dispenser dropper with a seal or gasket mechanism can also be used to achieve the same objectives of maintaining both the sterility and high PO₂ of the inventive composition.

Composition

In one embodiment, the invention is directed to an ophthalmic composition comprising a solution of an aqueous ophthalmic agent and oxygen, with the oxygen in gaseous form, and present in a concentration sufficient to substantially oxygenate the solution. The aqueous ophthalmic agent can either be artificial tears or another aqueous solution of an ophthalmic agent such as an antibacterial agent, an antiviral agent, a corticosteroid, a dilating agent, a glaucoma agent, a mydriatic, a cycloplegic, a non-steroidal anti-inflammatory drug (NSAID), ocular antihistamine, ocular decongestant, ocular MAST cell stabilizer, any commercially available eye drop or medication, or combinations thereof. In other words, by oxygenating any ocular medication or eye drop, the same benefits of the original medication or drop are expected to be retained, with the additional benefits listed above provided by a higher concentration of oxygen than in the non-oxygenated form of the product.

If artificial tears are utilized, the artificial tear will generally contain a diluting agent such as water, distilled water, or sterile water and a wetting agent. The wetting agent can include carboxymethylcellulose (CMC), hydroxypropyl methylcellulose (HPMC), glycerin, mannitol, polyvinyl alcohol (PVA), hydroxyethylcellulose, Carbopol, polyvinyl pyrrolidone, polyethylene glycol, dextran, hyaluronic acid, carbomer 940 (polyacrylic acid), or combinations thereof and is usually present in concentrations between about 0.001 and about 10 volume percent based on the total volume of the artificial tears.

The aqueous ophthalmic composition can also include a diagnostically or pharmaceutically acceptable amount of an excipient, depending on how the ophthalmic composition is to be utilized. The excipient is usually present in concentrations between about 0.01 and about 0.03 volume percent based on the total volume of the aqueous solution. The excipient is generally an antioxidant, sodium bisulfate, metabisulfate, hydrochloric acid, sodium hydroxide, or combinations thereof.

The aqueous ophthalmic composition can also include a diagnostically or pharmaceutically acceptable amount of an additive such as a buffer. The buffer is generally sodium bicarbonate or sodium lactate present in an amount sufficient to adjust the pH of the composition to between about 4.0 and 7.5.

Frequently, salts or other compounds are added to match the electrolyte balance of the eye. The salt is usually one or more of the following: calcium chloride, magnesium chloride, potassium chloride, sodium chloride, or sodium phosphate. To match the electrolyte balance, salts are generally present in a concentration between about 0.01 and about 4.0 volume percent based on the total volume of the aqueous ophthalmic agent.

Preservatives may also be included in the ophthalmic composition. Preservatives may be necessary to prevent bacteria growth and maintain the active agents within the ophthalmic composition. Suitable preservatives may include, but are not limited to, sodium perborate, benzalkonium chloride, purite, polyquaternium-1, sodium silver chloride complex, polyhexamethylene biguanide, sorbic acid, chlorobutanol, a stabilized oxyborate complex, or combinations thereof. The preservative concentration is generally between about 0.001 and about 1.0 volume percent based on the total volume of the solution.

Indications

Oxygenated ophthalmic compositions comprising an aqueous ophthalmic agent and oxygen can be administered to treat ocular pathology, particularly those complications and diseases caused by ocular hypoxia. The inventive composition may be used to treat ocular pathology including, but not limited to, contact lens intolerance, corneal neovascularization, dry eye syndrome, edema, abrasions, infiltrates, and infections, and symptoms including, but not limited to, itching, swelling, redness, irritation, blurred vision, discomfort, photophobia, and foreign body sensation.

Process of Manufacture

The oxygenated ophthalmic composition may be created by contacting the ophthalmic solution with gaseous oxygen, maintaining a high PO₂ environment over the entire production line so the resultant sealed containers have a solution with a high PO₂, filling a sealable container with the oxygenated aqueous ophthalmic composition, and then sealing the sealable container while maintaining an oxygen overpressure. With respect to high PO₂ or a high PO₂ environment, high is defined as encompassing any condition or amount that is higher than normal atmospheric oxygen conditions. The oxygen contact with the solution can be facilitated using conventional techniques for contacting gases with liquids such as bubbling, spraying, violently shaking, aerating, maintaining an oxygen overpressure, electrolysis, cold oxygen plasma, or combinations thereof.

The oxygenated ophthalmic composition can also be created filling a sealable container with an aqueous ophthalmic solution; filling the remainder of the sealable container with substantially pure oxygen gas; and sealing the single-dose vial to encapsulate the aqueous ophthalmic solution and oxygen gas.

Once the oxygenated ophthalmic composition is created, the oxygen level can be maintained by filling a sealable container with an oxygenated aqueous ophthalmic solution; filling the headspace of the sealable container with substantially pure oxygen gas; and sealing the sealable container. The oxygen level of the oxygenated ophthalmic composition can also be maintained by simply filling a sealable container with an oxygenated aqueous ophthalmic solution in the presence of an oxygen overpressure and sealing the container.

Composition Components

The inventive ophthalmic composition consists of an ophthalmic solution such as artificial tears, or an antibacterial agent, antiviral agent, corticosteroid, dilating agent, glaucoma agent, mydriatic, cycloplegic, non-steroidal anti-inflammatory drug (NSAID), ocular antihistamine, ocular decongestant, ocular MAST cell stabilizer, allergy drop, or any other suitable topical ophthalmic drop or medication or combination that has been substantially oxygenated for use in preventing or treating hypoxia of the anterior eye structures.

A preferred ophthalmic composition is oxygenated artificial tears containing one or more of the following components: a wetting agent (lubricant), buffer and/or acids and bases to adjust pH, purified water, and salts to match electrolytic balance. Additionally, the inventive solution may include hydrogels to increase cornea retention time and preservatives to increase shelf-life. Additional components may be added as desired without adversely affecting the function of the invention.

The inventive oxygenated solution may also contain wetting agents to increase retention time on the cornea and aid in matching the composition and texture of naturally produced tears. The wetting agent can include: carboxymethylcellulose (CMC), hydroxypropyl methylcellulose (HPMC), glycerin, mannitol, polyvinyl alcohol (PVA), hydroxyethylcellulose, Carbopol, polyvinyl pyrrolidone, polyethylene glycol, dextran, hyaluronic acid, carbomer 940 (polyacrylic acid) or other wetting agents that are conventional or will be developed as artificial technology improves. The concentration of the wetting agent in the inventive composition is generally between about 0.001% and about 10% by volume based on the total volume of the artificial tear solution. More preferably, the wetting agent concentration is between about 0.1% and about 1.0% by volume.

The inventive composition may also contain a diagnostically acceptable amount of excipients, additives, and/or buffers. Buffers and other excipients are typically added to adjust the pH of the composition, making it acceptable to the eye and maximizing the efficiency of the components. Buffers can include, but are not limited to, sodium bicarbonate and sodium lactate. Preferably, the pH of a composition instilled into the eye should be between about 4.0 to about 7.5. Excipients may include stabilizers and/or antioxidants to prevent drug degradation. Stabilizers may include sodium bisulfate, metabisulfate, hydrochloric acid, or sodium hydroxide, preferably in a concentration ranging from about 0.01% to about 0.03% by volume.

Salts are also used to match the electrolytic balance. Examples of salts include: calcium chloride, magnesium chloride, potassium chloride, sodium chloride, and sodium phosphate. Purified water is also used to increase overall volume, as it does not alter the solution and its constituents.

The additives described herein may be present in a diagnostically acceptable amount, pharmaceutically acceptable amount, or both, depending on the planned use for the ophthalmic composition.

The inventive composition contains dissolved oxygen. Because delivering as much oxygen as possible to the eye is desirable, the PO₂ of the inventive composition should be as high as feasible without causing deleterious side effects. The saturation level will vary depending on the temperature, salinity of the solution, and the barometric pressure. Strictly for exemplary purposes, some approximate saturation levels are: At 0° C., 4 PPT (parts per thousand) salinity, and 1050 mmHg, the saturation level may be about 2.5 mL oxygen per liter of solution; at 50° C., 9 PPT salinity, 380 mm Hg, the saturation level may be approximately 13.5 mL oxygen per liter of solution.

In order to prevent oxygen loss to the atmosphere, the inventive composition may be stored in single-dose vials with oxygen gas filling any space not utilized by the ophthalmic solution.

Any conventional ophthalmic solution can be oxygenated. This includes both over-the-counter ophthalmic solutions such as artificial tears, as well as prescription ophthalmic solutions. To oxygenate an ophthalmic solution, the conventional ophthalmic solution can be made in its conventional manner and then oxygenated as a subsequent step, or by placing the normal production line in a room with a high PO₂.

A standard ophthalmic solution can be subjected to any known method for enhancing oxygen-solution to make the inventive oxygenated ophthalmic solution. Such methods include, but are not limited to bubbling, spraying, aerating, or pressurizing headspace with gaseous O₂. Another method can include electrolysis of the aqueous portion of an ophthalmic solution to create oxygen gas within the solution. Cold oxygen plasma exposure can also be used to enhance the oxygen content of the ophthalmic solution. In bubbling, one uses an apparatus that has a tube with pure oxygen attached to a chamber with the ophthalmic solution. In spraying, one uses a canister with compressed oxygen and sprays the gas over the liquid. To aerate the solution, one places a beaker or other container with the solution in it inside a chamber that has only pure oxygen. Another method of oxygenation is to create an overpressure of oxygen above the ophthalmic composition. This could be achieved by oxygenating the air of a room, chamber, or other enclosed area, then placing the target solution within said enclosed area; after a given time to equilibrate, the ophthalmic composition will become oxygenated. This method can also be altered so that one simply injects oxygen (or uses another suitable method), in place of the air, into a vial of single-dose of the target ophthalmic composition, or replaces or fills the headspace with oxygen.

A preferred method of delivery to ensure retaining a high oxygen concentration in the ophthalmic solution is to use single-dose vials, closed to the atmosphere and possibly over-pressured with oxygen gas that are only opened immediately prior to use. For any of the above oxygenation methods, the only requirement is that the oxygen and ophthalmic solution be in substantial contact. Any method that creates suitable contact causes the oxygen to eventually equilibrate with the solution to create the inventive oxygenated ophthalmic solution.

Once the oxygenated ophthalmic solution is created, it is important to take appropriate measures to maintain the concentration of oxygen within the solution because if an oxygenated liquid is allowed to rest under atmospheric conditions, it will establish equilibrium with its surroundings and will thus result in a decreased oxygen saturation level. One method to prevent this equilibrium is to place the oxygenated ophthalmic solution in a sealed container to trap any oxygen that evaporates from the solution. The container can be sealed with any conventional method that will not allow significant amounts of oxygen or other gases to escape.

Simply sealing the container will result in significant amounts of oxygen evaporation because the solution will equilibrate with the atmospheric gas within the sealed container. In order to avoid this problem, the container storing the oxygenated ophthalmic solution (such as a single-dose vial) can be filled with substantially pure oxygen instead of air in order to maintain maximum reasonable saturation level by creating an equilibrium condition which does not favor the evaporation of oxygen from the solution while in storage. This is referred to as an overpressure of oxygen or filling the headspace with oxygen.

If containers that are repeatedly opened and closed are used to store the ophthalmic solution, the overpressure of oxygen will be lost after the first opening of the container. This problem can be solved by storing the ophthalmic solution in single-dose vials because the only evaporation occurs right before use of the ophthalmic solution resulting in a maximum obtainable oxygen concentration available to a standard patient. Because a new single-dose vial would be used per each ocular administration of the inventive composition, the high PO.sub.2 would be maintained, resulting in maximum oxygen delivery to the eye. Another method of maintaining maximum oxygen concentration when the ophthalmic solution is dispensed is to use a special eye drop dispenser containing a one way valve that does not allow oxygen and other gases to escape or enter during the administration of each eye drop.

Method of Administration and Usage

The inventive oxygenated ophthalmic compositions are administered on an as needed basis or as directed by the patient's opthalmologist or optometrist.

Most single-dose vials release between 0.02 mL to 0.04 mL of solution per “squeeze.” The inventive solutions are instilled as needed, which for most patients varies from between about once an hour to about once a day. The above dosage and administration instructions are for exemplary purposes only and do not in any way limit the scope of the inventive composition. The present ophthalmic compositions encompass any conventional or other suitable ophthalmic composition that is oxygenated as described herein. Additionally, the present compositions can be administered in any effective amount to prevent or treat problems associated with hypoxia of the anterior eye structures.

Kits

In yet another embodiment, the present invention encompasses a kit comprising an ophthalmic composition comprising a solution of an aqueous ophthalmic agent and oxygen. In yet another embodiment, the present invention comprises a kit that additionally includes patient instructions and other desired components, such as eye wipes or other components typically present in a surgical kit, for example. In yet another embodiment, the kit comprises at least one ophthalmic composition contained in a single-dose vial.

In as much as it may desirable to administer a combination of active compounds, for example, for the purpose of treating a particular disease or condition, it is within the scope of the present invention that two or more ophthalmic compositions, at least one of which comprises a solution of an aqueous ophthalmic agent and oxygen, may conveniently be combined in the form of a kit suitable for co administration of the compositions.

### EXAMPLE 1

A tube was connected from a medical grade oxygen tank (UN1072; CAS Number: 80937-33-3) to a bottle of Allergan Refresh Plush Lubricant Eye props (3 mL). This is an example of a commercially available conventional artificial tear. The oxygen was bubbled through the eye drops at a rate of 8 liters per minute (1 pm) for 10 seconds, after which the bottle was quickly capped to prevent loss of oxygen. Next, the bottle was shaken and inverted to increase the oxygen concentration within the eye drops.

To preliminarily determine the effectiveness of the oxygenated eye drops, the inventive composition was administered to 10 patients. Each subject had one eye designated as a test eye and one eye designated as a control eye. Test and control eye numbers were randomized using a random number generator. The test eye received one drop of the inventive composition (oxygenated artificial tear solution) and the control eye received one drop of the standard (non-oxygenated artificial tear solution) at the same time. Five minutes after the bilateral administration, subjects were asked to assess their subjective assessment of hypoxia alleviation by comparing the following attributes: comfort, smoothness, coolness, and also to choose the eye drop that they preferred (forced choice).

Ten out of 10 patients reported that the oxygenated eye drop felt “smoother” and “more comfortable” than the control drop in the contra lateral eye. Seven of 10 said the oxygenated drop felt “cooler” and 7 out of 10 said they “preferred” the oxygenated drop over the control. Of the 3 postoperative patients (2 post-LASEK and 1 post-LASIK), all 3 preferred the oxygenated drop and responded that it felt cooler, smoother, and more comfortable than control.

Based on the results obtained from the above pilot study, a larger study was performed between September 2006 and June 2007 on 125 pre-operative patients who were about to undergo laser vision correction, but did not have any prior ophthalmic history or surgery. The methods in the larger study were the same as in the pilot study.

P-values were calculated for each descriptor that patients were asked to compare (preference, coolness, smoothness, comfort). Since patients were asked to choose one of the drops for each category in a Boolean fashion (either the oxygenated drop or the regular drop), a 1-proportion z-test was used to determine p-values with Ho: p₀=0.50, and Ha: p₀>0.50. The proportions were defined as the number of patients who selected the oxygenated drop for a particular category divided by the total number of patient responses for that particular category.

The p-values for the 125-patient study are as follows: preference=<0.001; coolness=<0.001; smoothness=0.021; comfort=0.014. These values clearly indicate a statistically significant preference for the oxygenated drops for all four assessed variables, including patient preference.

### Conclusions

The present data support the rationale behind, composition of, and various methods of use and manufacture of inventive oxygenated ophthalmic solutions for preventing, ameliorating, and/or treating ocular pathology and symptomatology associated with hypoxia of the anterior eye structures, as well as various kits useful as described herein. A study of 125 patients demonstrated with statistical significance that the use of the inventive composition as embodied in an oxygenated artificial tear solution was both beneficial to, and preferred by subjects over standard, non-oxygenated tears. It is understood that various omissions, substitutions, and changes in the form and details of the inventive composition, in its manufacture and administration may be made by those skilled in the art without departing from the spirit and scope of the invention. Features, aspects, and steps in any disclosed embodiment can generally be employed in any other embodiment with equal advantage and is intended within the scope of the invention.

The present invention is not to be limited in scope by the specific embodiments described herein. Indeed, various modifications of the invention in addition to those described herein will become apparent to those skilled in the art from the foregoing description and the accompanying FIGURES. Such modifications are intended to fall within the scope of the appended claims.

It is further to be understood that all values are approximate, and are provided for description. Patents, patent applications, publications, product descriptions, and protocols are cited throughout this application, the disclosures of which are incorporated herein by reference in their entireties for all purposes.

1. A method of treating an ocular condition comprising administering an ophthalmic composition comprising a solution of an aqueous ophthalmic agent containing oxygen via topical ocular administration to a patient in need thereof, wherein the composition is administered from a single-dose container.

2. The method of claim 1 wherein the ocular condition is ocular hypoxia.

3. The method of claim 1 wherein the ocular condition is contact lens intolerance syndrome.

4. The method of claim 1 wherein the ocular condition is corneal neovascularization.

5. The method of claim I wherein the ocular condition is one or more corneal abrasion.

6. The method of claim I wherein the ocular condition is hypoxia of one or more of the anterior eye structures.

7. The method of claim 1 wherein the ocular condition is ocular pathology.

8. The method of claim 2, wherein the administration of said solution is from a single-dose container wherein any space not utilized by the aqueous ophthalmic agent is filled with oxygen gas.

9. The method of claim 8 wherein the aqueous ophthalmic agent contains approximately 13.5 ml oxygen per liter of solution.

10. A method of treatment of eye irritation caused by an eye procedure, comprising administering an ophthalmic composition comprising a solution of an aqueous ophthalmic agent containing oxygen via topical ocular administration to a patient in need of such treatment.

11. The method of claim 10, wherein the eye procedure is selected from the group consisting of LASEK and LASIK surgery.

12. The method of claim 10, wherein the aqueous ophthalmic agent is administered from a single dose container.

13. The method of treatment of ocular hypoxia, comprising administering an ophthalmic composition comprising an ophthalmic agent containing oxygen via topical ocular administration to a patient in need thereof wherein the ophthalmic agent is selected from the group consisting of an antibacterial agent, an antiviral agent, a corticosteroid, a dilating agent, a glaucoma agent, a mydriatic, a cycloplegic, a non-steroidal anti-inflammatory drug (NSAID), an ocular antihistamine, an ocular decongestant, an ocular MAST cell stabilizer, an allergy agent, and combinations thereof.

14. The method of claim 13, wherein the ocular hypoxia is intraocular hypoxia.

15. The method of claim 13, wherein the hypoxia is of one or more of the anterior eye structures.
